(19) United States
(12) Patent Application Publication
(10) Pub. No.: US 2007/0017151 A1
(43) Pub. Date: Jan. 25, 2007

(54) Nucleophilic Acyl Substitutions of Acids or Esters Catalyzed by Oxometallic Complexes, and the Applications in Fabricating Biodiesel

(75) Inventor: Chien-Tien Chen, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN NORMAL UNIVERSITY, Taipei (TW)

(21) Appl. No.: 11/459,007

(22) Filed: Jul. 20, 2006

(30) Foreign Application Priority Data
Jul. 22, 2005 (TW) 094124839

(51) Int. Cl. C10L 1/22; C07C 51/43

(52) U.S. Cl. 44/335; 554/174

(57) ABSTRACT

The present invention discloses a method of nucleophilic acyl substitution (NAS) of carboxylic acids or esters (hereinafter acids/esters) catalyzed by oxometallic complexes. According to the mentioned method, NAS reactions between acids/esters (R¹COOH/R¹—COO—R²) and protic nucleophile (R³-AH) can be catalyzed by oxxmetallic complexes, wherein A stands for O, S, or NH. The general formula of the mentioned oxometallic complexes is MOₘL¹_{y}L²_{z}, wherein M is selected from IVB, VB, VIB or actinide groups, m, y, z are integers, and m, y≧1, z≧0. A general catalytic equation is shown as follows:

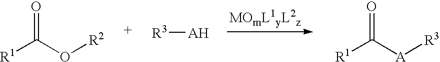

### BACKGROUND OF THE INVENTION

1. Field of the Invention

The present invention is generally related to a method of catalytic nucleophilic acyl substitutions, and more particularly to a method of nucleophilic acyl substitutions of acids or esters catalyzed by oxometallic complexes and its applications in fabricating biodiesel.

2. Description of the Prior Art

Direct esterification reactions are extensively applied in industry. In general, commercial ester-containing products comprise varnish, solvents, essence, elasticizers, resin curing agents, medicine synthetic intermediates and so forth. Conventional esterification reactions use acids and excess amount of alcohols as the raw materials in the presence of Bronsted acid catalysts, such as sulfuric acid, boric acid, or hydrochloric acid to accelerate the esterifiction reactions. However, it has the disadvantages of dealing with subsequent waste wate and the process equipments need anti-corrosive treatment due to the addition of strong acids. More critically, the alcohols can not have acid-sensitive functional groups like tetrahydropyranyl ethers, silyl ethers, and acetonides. In addition, it has been widely reported that Sn(II) and Sn (IV) species can be used to catalyze the esterification reactions. Although the catalytic performance is satisfactory, they are highly neuro-toxic which result in potential damages to operator's health and to the environment.

In addition, trans-esterification reactions play an important role in synthetic organic chemistry. Trans-esterification reactions can be applied not only in the synthesis of various esters but also in the industrial processes of dyes, suntan lotions (UV absorbers), preservatives, and etc. In general, the catalysts for trans-esterification reactions comprise (1) Bronsted acids (H₃PO₄, H₂SO₄, HCl) and organic acid (p-TSA); (2) alkaline oxides (LiOR, NaOR, and KOR) or alkaline earth oxides (ROMgBr); (3) Lewis bases (4-N,N-dimethylaminopyridine, DBU, imidazolinium carbenes); (4) Lewis acids (BX₃, AlCl₃, Al(OR)₃); (5) tin-containing compounds (Bu₃SnOR, SnCl₂, Sn(O₂CR)₂, Bu₂SnO) palladium salts, and titanium alkoxide/titanium chloride (Ti(OR)₄, Ti(OR)₂(acac)₂/TiCl₄). Although the above catalytic systems can provide high conversion rate, the following essential problems remain to be resolved: (1) excess amount of alcohols or esters needed; (2) high dosages in catalyst loadings; (3) catalyst by-products are not water-soluble and toxic to the environment; (4) limited functional group compatability.

On the other hand, biodiesel is a substitute for petroleum diesel, representing one of the major fuel substitutes researched by developed countries. The production method of biodiesel uses recycled biomass sources, such as vegetable oil or animal oil, as the raw materials to chemically fabricate into 00the biodiesel. Because biodiesel has renewability, biodegradability, and avirulence. In addition, the concentration of pollutants from burning biodiesel is significantly lower than that from burning the petroleum diesel. It is also one of the renewable energy sources that meet the requirements of worldwide sustainable development and environmental protection policy. The methods for fabricating biodiesel typically have four different routes. At present, the most commonly used method requires triglyceride from vegetable oil and short-chained alcohols (C1-C5) to perform a trans-esterification reaction to generate an alkyl ester, i.e. biodiesel, together with glycerol by-product. The general equation is as follows: 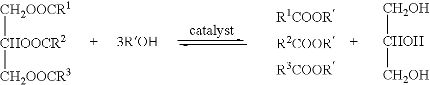
Alcohols used in fabricating biodiesel normally comprise methanol, ethanol, propanol, butanol, and pentanol in which methanol and ethanol are most extensively used. Especially, because methanol has the merits of lower cost and stable physical and chemical properties, methanol constitutes the major source in biodiesel fabrication. Therefore, fatty acid methyl esters formed by the trans-esterification reaction represent the most common biodiesel.

The reaction rate of the trans-esterification catalyzed by base (hereafter referring to as base process) is 4000 times faster than that catalyzed by acid. Therefore, base process is extensively used in the commercial processes. However, if crude oil comprises excess amount of free fatty acids, free fatty acids react with the base catalyst to form soap in the base process. Therefore, the use of crude oil results in disadvantages, such as consuming catalyst, reducing catalytic efficiency, and increasing the viscosity of the reaction mixture. In order to solve the above mentioned problems, the crude oil containing excess amount of fatty acids are transformed into methyl esters with acid catalysis in the first reactor with the operational temperature near the boiling point of methanol (60° C.) for 40 minutes. Then, a base catalyzed reaction under similar conditions in the second reactor is performed to fabricate biodiesel and generate glycerol by-product.

In light of the above-mentioned problems, a new neutral, wter-tolerant catalyst is still in great demand to fulfill the requirements of non-corrosive or even neutral property, low toxicity, enviromental protection. This remains an important research aspect in the industrial practical applications.

### SUMMARY OF THE INVENTION

In view of the above background and to fulfill the requirements of the green industry, a new method of nucleophilic acyl substitutions of acids or esters (hereinafter acids/esters) catalyzed by oxometallic complexes and its applications in fabricating biodiesel are invented.

One subject of the present invention is to provide a new method of nucleophilic acyl substitutions of acids/esters catalyzed catalyzed by oxometallic complexes. The method uses oxometallic complexes to catalyze the nucleophilic acyl substitution reactions between acids/esters and protic nucleophilic reagents. The method can be readily operated under mild reaction conditions with high chemical selectivity and excellent chemical yields. In addition, the oxometallic complexes provided by the present invention display the characteristics of long-term activity, and high water and air compatibilities. Thus, the production cost is significantly reduced. Furthermore, the oxometallic complexes can be recycled after the nucleophilic acyl substitution reaction and the recycled catalysts still maintain excellent catalytic function. Therefore, the method according to the present invention has not only the economic advantages for industrial applications but also environmental friendliness.

Another subject of the present invention is to provide a method for fabricating biodiesel catalyzed by oxometallic complexes. Compared to the current technique that requires two reactors with pretreatment of any free fatty acids in the crude oil, the method according to the invention can be operated in a one-pot reaction, which performs biodiesel formation from free fatty acid (direct esterification) and and triglyceride (trans-esterification) simultaneously. Thus, the method is potentially highly valuable in industrial applications.

Accordingly, the present invention discloses a method of nucleophilic acyl substitution (NAS) of acids/esters catalyzed by oxometallic complexes. According to the mentioned method, NAS reaction between acids/esters (R¹COOH/R¹—COO—R²) and protic nucleophile (R³-AH) can be catalyzed by oxometallic complexes, wherein A stands for O, S, or NH. The general formula of the mentioned oxometallic complexes is MOₘL¹_{y}L²_{z} wherein m and y are integers of greater than or equal to 1 and z is an integer of greater than or equal to zero. A general catalytic equation is shown as follows: 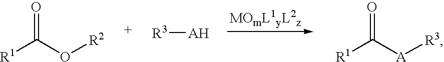
wherein M is selected from IVB, VB, VIB or actinide groups.

### DESCRIPTION OF THE PREFERRED EMBODIMENTS

What is probed into the invention is a method of nucleophilic acyl substitutions of acids/esters catalyzed by oxometallic complexes and its applications in fabricating biodiesel. Detailed descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater details in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In the first embodiment of the present invention, a method of nucleophilic acyl substitutions of acids/esters catalyzed by oxometallic complexes is provided. At first, an acid/ester (R¹COOH/R¹—COO—R²) is provided. The NAS reaction between acids/esters (R¹COOH/R¹—COO—R²) and protic nucleophile (R³-AH) is catalyzed by oxometallic complexes, wherein A stands for O, S, or NH. The general formula of the mentioned oxometallic complex is MOₘL¹_{y}L²_{z} wherein m and y are integers of greater than or equal to 1 and z is an integer of greater than or equal to 0. A general NAS reaction equation is as follows: 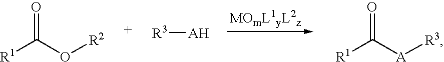
wherein M is selected from IVB, VB, VIB or actinide groups. The above R¹ and R³ comprise one selected from the group consisting of the following: linear, branched, or cyclic alkyl moiety; linear, branched, or cyclic alkyl moiety including one or more than one substituted moiety selected from the group consisting of alkene, alkyne, halide moiety, alkoxy, siloxy, ketone, alcohol, thioether, carbamate or amino moiety; aromatic group; heterocyclic group; multiple fused ring group; and, multiple fused ring group with heteroatoms. Besides, the R² is H or C₁-C₅ alkyl group. On the other hand, the above L¹ comprises one selected from the group consisting of the following: OTf, X, RC(O)CHC(O)R, OAc, OEt, O-iPr, butyl, in which X comprises halogen elements. The above L² comprises one selected from the group consisting of the following: H₂O, CH₃OH, EtOH, THF, CH₃CN,

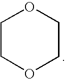

In a preferred embodiment of this embodiment, the metal of the oxometalic complex is an IVB transition metal element. When m=1, y=2, the preferred metal further comprises one selected from the group consisting of the following: titanium (Ti), zirconium (Zr), and hafnium (Hf). To explain the reaction of this embodiment in detail, preferred reactions according to this embodiment are shown in the following.

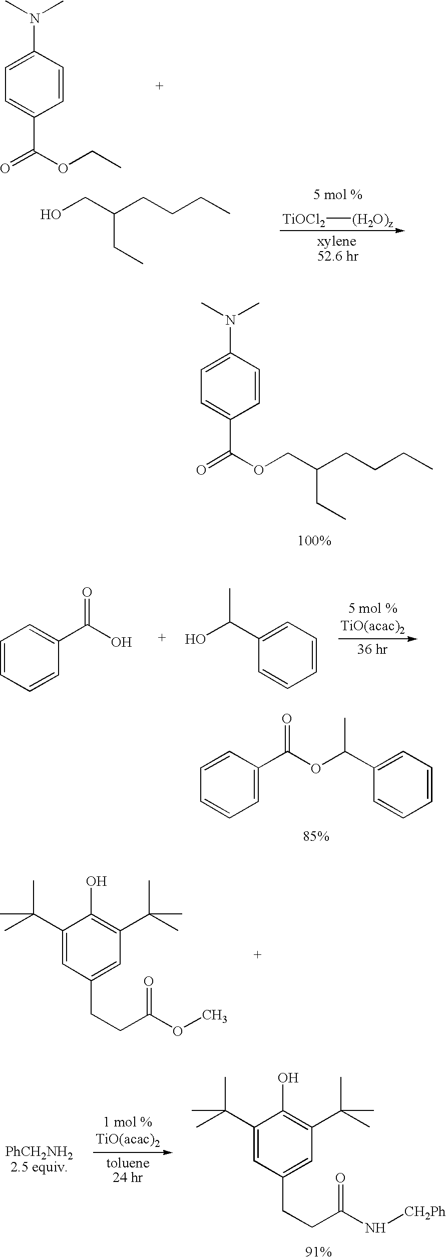

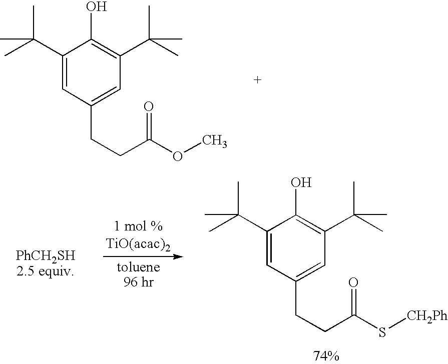

In another preferred embodiment of this embodiment, the metal of the oxometallic complex is a VB transition metal element. As m=1, y=2 or as m=1, y=3, the preferred metal further comprises vanadium (V) or niobium (Nb). To explain the reaction of this embodiment in detail, a preferred reaction according to this embodiment is shown in the following.

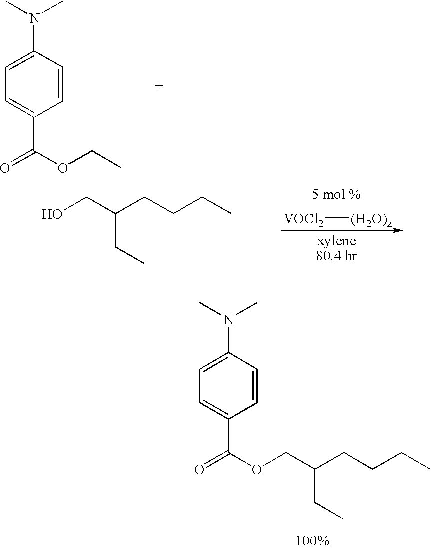

In another preferred embodiment of this embodiment, the metal of the oxometalic complex is a VIB transition metal element. As m=1, y=4 or as m=1=2, y=2. The preferred metal further comprises molybdenum (Mo), tungsten (W), or chromium (Cr). To explain the reaction of this embodiment in detail, a preferred reaction according to this embodiment is shown in the following.

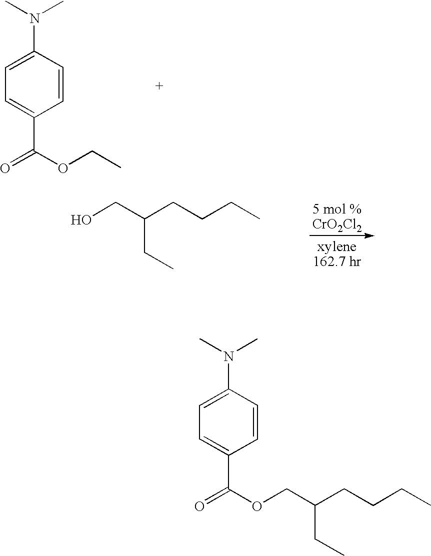

In another preferred embodiment of this embodiment, the metal of the oxometallic complex is an actinide transition metal element. As m=2, y=2 and the preferred metal further comprises uranium (U).

### EXAMPLE

Procedure Fior the Nicleophilic Acyl Substitution Reaction:

A two-necked, 50-mL flask with a stirring bar is equipped with a Dean-Stark trap. The flask is vacuum dried by flame and thereby is slowly cooled to room temperature, and is flushed with nitrogen gas. About 3 mL of water is placed inside the trap. 5 mmol of esters or carboxylic acids and 5 mmol of protic nucleophiles (e.g., alcohols, thioesters, or amines) are precisely measured. Then, 10 mL of nonpolar solvent, such as high boiling (cyclo)alkanes, ethers (anisole, dioxane, or DME), haloalkanes (e.g., chloroform or carbon tetrachloride (CCl₄), or arenes (e.g., benzene, toluene, ethylbenzene, or xylene) is added. The reaction content in the flask is stirred to become homogeneous while heated up to the refluxing temperature with removal of water. After having been refluxed for 30 minutes, the reaction mixture is then cooled to room temperature. Catalyst loading typically in 0.1-10 mol % is measured and placed in the reaction flask. The reaction flask is again heated up to the refluxing temperature. After the reaction is complete, the reaction flask is then cooled to room temperature and quenched by adding cold aqueous NaHCO₃ solution (5 mL). The resulting separated organic layer is dried by magnesium sulfate, filtered, and evaporated. The crude product can be provided with reasonably good purity. Pure product can be obtained by induced precipitation or by column chromatography.

Process of Recycling Catalyst:

The reaction content in the flask is then heated to the refluxing temperature with removal of water. After the reaction is complete, the reaction flask is then cooled to room temperature. Part of solvent is evaporated to concentrate the reaction solution.

A two-necked, 50-mL flask with a stirring bar is equipped with a Dean-Stark trap. The flask is then vacuum dried by flame and thereby is slowly cooled to room temperature, and is flushed with nitrogen gas. About 3 mL of water is placed inside the trap. 5 mmol of esters or carboxylic acids and 5 mmol of protic nucleophiles (e.g., alcohols, thioesters, or amines) are precisely measured. Then, 10 mL of anhydrous nonpolar solvent mentioned above is added. The reaction content in the flask is then heated to the refluxing temperature with removal of water. After having been refluxed for 30 minutes, the reaction flask is then cooled to room temperature. Catalyst with proper loading, such as 0.1-10 mol %, is precisely measured and placed in the reaction flask. The reaction flask is again heated to the refluxing temperature. After the reaction is complete, the reaction flask is then cooled to room temperature and the reaction is quenched by adding 25 mL of ice water. The resulting separated organic layer is dried by magnesium sulfate, filtered, and evaporated. The crude product can be provided with reasonably good purity. Pure product can be obtained by induced precipitation or by column chromatography. Water is removed from the resulting separated aqueous layer by a rotary evaporator. Then, the crude residue is further dried in vacuo for 2 hours to obtain recycled oxometallic complex (recovery yield >95%).

For example, the product 2-ethyl-1-hexyl 4-dimethylamino-benzoate has the following spectroscopic and analysis data:

Data: M.W. 277.40 (C₁₇H₂₇NO₂); ¹H NMR (400 MHz, CDCl₃) 7.92 (d, J=9.0, 2H, HC(3,5)), 6.65 (d, J=9.1, 2H, HC(2,6)), 4.24-4.15 (2H, HₐH_{b}C(9)), 3.19 (s, 6H, N(CH₃)₂), 1.73-1.65 (m, 1H, HC(10)), 1.53-1.26 (m, 8H, HC(11-13, 15)), 0.95 (t, J=7.5, 3H, CH₃), 0.93 (t, J=7.8, 3H, CH₃); ¹³C NMR (100 MHz, CDCl₃) 166.95 (C═O), 153.11 (C(4)), 131.03 (C(3,5)), 117.31 (C(1)), 111.57 (C(2,6)), 66.35 (OCH₂), 39.83(N(CH₃)₂), 38.93 (C(10)), 30.56 (C(11)), 28.91 (C(12)), 23.96 (C(15)), 22.87 (C(13)), 13.91 (CH₃), 11.0 (CH₃); MS (70 eV) 277 (M⁺, 100), 165 (66), 148 (70); IR (CH₂Cl₂) 3064 (s), 2964 (s), 1695 (s), 1607 (s), 1528 (s), 1427 (s), 1288 (s), 1245 (s), 1185 (s), 1113 (s); TLC R_{f} 0.4 (EtOAc/hexane, 1/8); High-resolution MS calcd for C₁₇H₂₇NO₂: 277.2042, found: 277.2042.

In the second embodiment of the present invention, a method for fabricating biodiesel is disclosed. At first, triglyceride-containing crude oil and a first alcohol R⁴-AH are mixed in a given solvent. The first alcohol with number of carbons less than 4. Next, oxometalic complex is added into the reaction mixture. The trans-esterification reaction between the triglyceride-containing crude oil and the first alcohol R⁴-AH catalyzed by the oxometallic complex is performed to form the biodiesel. The reaction temperature of the trans-esterification is in a range of 60 to 300° C. The oxometallic complex has the general formula MOₘL¹_{y}L²_{z} in which m and y are integers of greater than or equal to 1 and z is an integer of greater than or equal to zero. The metal M comprises one selected from a group consisting of the following: IVB, VB, VIB, and actinide groups. On the other hand, the above L¹ comprises one selected from the group consisting of the following: OTf, X, RC(O)CHC(O)R, OAc, OEt, O-iPr, butyl, in which X comprises halogen elements. The above L² comprises one selected from the group consisting of the following: H₂O,

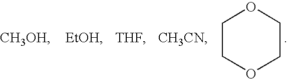

In this embodiment, M comprises the following four groups: IVB, VB, VIB, actinide groups. The m and y depend on the classification of the metal M. For example, [1] as the metal M comprises an IVB group transition metal element and m=1, y=2 and the preferred metal M further comprises one selected from the group consisting of the following: titanium (Ti), zirconium (Zr), and hafnium (Hf); [2] as the metal M comprises a VB group transition metal element and m=1, y=2 or as m=1, y=3 and the preferred metal M further comprises vanadium (V) or niobium (Nb); [3] as the metal M comprises a VIB group transition metal element and m=1, y=4 and the preferred metal M further comprises molybdenum (Mo), tungsten (W), or chromium (Cr); [4] as the metal M comprises an actinide group transition metal element and m=2, y=2 and the preferred metal M further comprises uranium (U).

In a preferred embodiment of this embodiment, the method for fabricating biodiesel further comprises performing a direct esterification reaction by using the oxometallic complex to catalyze the reaction between free fatty acid R⁵—COOH in the crude oil and a second alcohol R⁶—OH to form an intermediate oil. The first alcohol can be the same as or different from the second alcohol. The oxometallic complex continues catalyzing the first alcohol R⁴—OH or the second alcohol R⁶—OH to react with the intermediate oil so as to form the biodiesel. Compared to the commercial technique that requires two reactors with pretreatment of any free fatty acids in the crude oil, the method according to the invention can be operated in a one-pot reaction, which performs biodiesel formation from free fatty acid (direct esterification) and and triglyceride (trans-esterification) simultaneously. Thus, the method is commercially highly valuable. In addition, in this embodiment, the direct esterification and the trans-esterification can be performed in a high pressure reactor to increase the reaction temperature and reaction efficiency.

In the above embodiment, the invention invokes the oxometallic complex to catalyze the nucleophilic acyl substitutions of acids/esters by alcohols. Because the catalytic method exerted by oxometallic complex provided by the present invention is in a simple manner and has lower process cost, easy recovery of catalyst from the reaction product, high water compatibility, high chemical selectivity, and excellent chemical yields, the present invention has the economic advantages for industrial applications. Furthermore, the method according to the invention is a one-pot reaction and performs biodiesel formation from free fatty acid (direct esterification) and and triglyceride (trans-esterification) simultaneously. Thus, the method is commercially highly valuable.

To sum up, the present invention discloses a method of nucleophilic acyl substitutions of acids/esters catalyzed by oxometallic complexes. At first, an acid/ester (R¹COOH/R¹—COO—R²) is provided. The NAS reaction between acids/esters (R¹COOH/R¹—COO—R²) and protic nucleophile (R³-AH) is catalyzed by a given oxometallic complex, wherein A stands for O, S, or NH. The general formula of the mentioned oxometallic complex is MOₘL¹_{y}L²_{z} wherein m and y are integers of greater than or equal to 1 and z is an integer of greater than or equal to zero. A general NAS reaction equation is as follows: 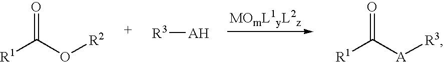
wherein M is selected from IVB, VB, VIB or actinide groups.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method of nucleophilic acyl substitutions of acids or esters (hereinafter as acids/esters) catalyzed by oxometallic complexes, comprising:
providing an acid/ester (R¹—COOH/R¹—COO—R²); and,
catalyzing a nucleophilic acyl substitution between said acid/ester and a protic nucleophilic reagent R³-AH by an oxometallic complex;
wherein A stands for O, S, or NH, said oxometallic complex has the general formula MOₘL¹_{y}L²_{z} in which m and y are integers of greater than or equal to 1 and z is an integer of greater than or equal to zero, and said nucleophilic acyl substitution has the following general equation: 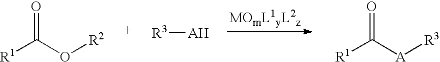
wherein said metal M of said oxometallic complexes comprise one selected from a group consisting of the following: IVB, VB, VIB and actinide groups.

2. The method of nucleophilic acyl substitutions of acids/esters catalyzed by oxometallic complex according to claim 1, wherein said R¹ and R³ comprise one selected from the group consisting of the following: linear, branched, or cyclic alkyl moiety; linear, branched, or cyclic alkyl moiety including one or more than one substituted moiety selected from the group consisting of alkene, alkyne, halide, alkoxy, siloxy, ketone, alcohol, thioether, carbamate or amino moiety; aromatic moiety; heterocyclic moiety; multiple fused ring group; and, multiple fused ring group with heteroatoms.

3. The method of nucleophilic acyl substitutions of acids/esters catalyzed by oxometallic complexes according to claim 1, wherein said R² is H or C₁-C₅ alkyl group.

4. The method of nucleophilic acyl substitutions of acids/esters catalyzed by oxometallic complexes according to claim 1, wherein said L¹ comprises one selected from the group consisting of the following: OTf, X, RC(O)CHC(O)R, OAc, OEt, O-iPr, butyl in which X comprises halogen elements.

5. The method of nucleophilic acyl substitutions of acids/esters catalyzed by oxometallic complexes according to claim 1, wherein said L² comprises one selected from the group consisting of the following:

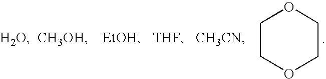

6. The method of nucleophilic acyl substitutions of acids/esters catalyzed by oxometallic complexes according to claim 1, wherein y=2 as said metal M includes an IVB transition metal element and m=1.

7. The method of nucleophilic acyl substitutions of acids/esters catalyzed by oxometallic complexes according to claim 6, wherein said metal M further comprises one selected from the group consisting of the following: titanium (Ti), zirconium (Zr), and hafnium (Hf).

8. The method of nucleophilic acyl substitutions of acids/esters catalyzed by oxometallic complexes according to claim 1, wherein y=2, as said metal M comprises a VB transition metal and m=1.

9. The method of nucleophilic acyl substitutions of acids/esters catalyzed by oxometallic complexes according to claim 8, wherein said metal M further comprises vanadium (V) or niobium (Nb)

10. The method of nucleophilic acyl substitutions of acids/esters catalyzed by oxometallic complexes according to claim 1, wherein y=3 as said metal M comprises a VB transition metal and m=1.

11. The method of nucleophilic acyl substitutions of acids/esters catalyzed by oxometallic complexes according to claim 10, wherein said metal M further comprises vanadium (V) or niobium (Nb).

12. The method of nucleophilic acyl substitutions of acids/esters catalyzed by oxometallic complexes according to claim 1, wherein y=4 as said metal M comprises a VIB transition metal and m=1.

13. The method of nucleophilic acyl substitutions of acids/esters catalyzed by oxometallic complexes according to claim 12, wherein said metal M further comprises molybdenum (Mo), tungsten (W), or chromium (Cr).

14. The method of nucleophilic acyl substitutions of acids/esters catalyzed by oxometallic complexes according to claim 1, wherein y=2 as said metal M comprises a VIB transition metal and m=2.

15. The method of nucleophilic acyl substitutions of acids/esters catalyzed by oxometallic complexes according to claim 14, wherein said metal M further comprises molybdenum (Mo), tungsten (W), or chromium (Cr).

16. The method of nucleophilic acyl substitutions of acids/esters catalyzed by oxometallic complexes according to claim 1, wherein y=2 as said metal M comprises an actinide transition metal and m=2.

17. The method of nucleophilic acyl substitutions of acids/esters catalyzed by oxometallic complexes according to claim 16, wherein said metal M further comprises uranium (U).

18. A method for fabricating biodiesel:
providing and mixing triglyceride-containing crude oil and a first alcohol R⁴-AH to form a solution mixture; and,
adding a given oxometallic complex into said solution mixture wherein said the oxometallic complex has the general formula MOₘL¹_{y}L²_{z} in which m and y are integers of greater than or equal to 1 and z is an integer of greater than or equal to zero and said metal M of said oxometallic complexes comprise one selected from a group consisting of the following: IVB, VB, VIB, and actinide groups;
performing a trans-esterification reaction between said triglyceride-containing crude oil and said first alcohol R⁴-AH catalyzed by said oxometallic complex to form the biodiesel.

19. The method for fabricating biodiesel according to claim 18, wherein said first alcohol is an alcohol with number of carbons less than 4.

20. The method for fabricating biodiesel according to claim 18, wherein said L¹ comprises one selected from the group consisting of the following: OTf, X, RC(O)CHC(O)R, OAc, OEt, O-iPr, butyl in which X comprises halogen elements.

21. The method for fabricating biodiesel according to claim 18, wherein said L² comprises one selected from the group consisting of the following:

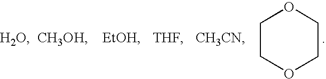

22. The method for fabricating biodiesel according to claim 18, wherein y=2 as said metal M comprises an IVB transition metal element and m=1.

23. The method for fabricating biodiesel according to claim 22, wherein said metal M further comprises one selected from the group consisting of the following: titanium (Ti), zirconium (Zr), and hafnium (Hf).

24. The method for fabricating biodiesel according to claim 18, wherein y=2 as said metal M comprises a VB transition metal and m=1.

25. The method for fabricating biodiesel according to claim 24, wherein said metal M further comprises vanadium (V) or niobium (Nb).

26. The method for fabricating biodiesel according to claim 18, wherein y=3 as said metal M comprises a VB transition metal and m=1.

27. The method for fabricating biodiesel according to claim 26, wherein said metal M further comprises vanadium (V) or niobium (Nb)

28. The method for fabricating biodiesel according to claim 18, wherein y=4 as said metal M comprises a VIB transition metal and m=1.

29. The method for fabricating biodiesel according to claim 28, wherein said metal M further comprises molybdenum (Mo), tungsten (W), or chromium (Cr).

30. The method for fabricating biodiesel according to claim 18, wherein y=2 as said metal M comprises a VIB transition metal and m=2.

31. The method for fabricating biodiesel according to claim 30, wherein said metal M further comprises molybdenum (Mo), tungsten (W), or chromium (Cr).

32. The method for fabricating biodiesel according to claim 18, wherein y=2 as said metal M comprises an actinide transition metal and m=2.

33. The method for fabricating biodiesel according to claim 32, wherein said metal M further comprises uranium (U).

34. The method for fabricating biodiesel according to claim 18, wherein the reaction temperature of said trans-esterification is greater than 60° C.

33. The method for fabricating biodiesel according to claim 18, further comprises: performing a direct esterification reaction by using said oxometallic complex to catalyze free fatty acid R⁵—COOH in said crude oil to react with a second alcohol R⁶—OH to form an intermediate oil for said oxometallic complex to continue catalyzing said first alcohol R⁴—OH or said second alcohol R⁶—OH to react with said intermediate oil so as to form the biodiesel.

36. The method for fabricating biodiesel according to claim 35, wherein said first alcohol R⁴—OH is the same as said second alcohol R⁶—OH.
